(19) United States
(12) Patent Application Publication
(10) Pub. No.: US 2008/0044808 A1
(43) Pub. Date: Feb. 21, 2008

(54) West nile virus antigen and assay

(76) Inventor: Qian-Li Song, Mississauga (CA)

Correspondence Address: KLAUBER & JACKSON, 411 HACKENSACK AVENUE, HACKENSACK, NJ, 07601, US

(21) Appl. No.: 11/504,228

(22) Filed: Aug. 15, 2006

(51) Int. Cl. C12Q 1/70; C07H 21/04; C12N 15/86; C07K 14/08

(52) U.S. Cl. 435/5; 435/69.3; 435/325; 435/456; 530/350; 536/23.72

(57) ABSTRACT

The present invention provides for a polypeptide comprising a fragment of the West Nile Virus E protein, wherein the E protein fragment consists of residues 1-428. The invention also provides for polynucleotides encoding this protein and host cells transformed genetically to express this protein. More particularly, the invention provides for assays and diagnostic kits for the detection of West Nile Virus E protein antibody that circulates in the blood of patients thought to be afflicted with West Nile disease.

### FIELD OF THE INVENTION

This invention relates to assays and reagents useful to diagnose infection by West Nile Virus.

### BACKGROUND OF THE INVENTION

Mosquito-mediated infection by the flavivirus known as West Nile Virus (WNV) is a health risk now prevalent in North America. Infection by West Nile Virus can be detected by analyzing a sample of body fluid extracted from a subject for the presence of circulating antibody that binds with WNV antigens including particularly the envelope protein, or E protein. The WNV E protein is a 501 residue protein, as expressed by WNV strain NY-99-flamingo-382-99. In general, the flavivirus E proteins comprise at least three distinct domains designated, from the N-terminus to the C-terminus, as domains A, B and C (see Mandl et al, J. Viorl., 1989, 63(2):564-571). The relative contributions of each domain to the overall antigenicity of the E protein have been investigated, with a view to identifying the preferred antigen for use in vaccines and for in vitro diagnostic tests that detect circulating antibody as a marker of WNV infection.

The C-terminal part of the extracellular region of the WNV E protein, which may be referred to as domain D, also appears to have a significant role in antigenicity, and comprises a region of significant hydrophobicity. The latter characteristic generally prevents the production of the E protein in yield sufficient for its use as a diagnostic reagent or vaccine component. There is accordingly an interest in identifying a WNV antigen that possess both the ease of production and the antigenicity required for commercial use. Various fragments of the E protein have been studied.

WO96/37221 describes a flavivirus E protein fragment termed “80% E”. In the context of WNV, this is a C-terminally truncated E protein incorporating residues 1-395. There is also mention of 60% E, i.e., residues 1-237. Similarly, WO99/06068 describes and claims antigens that are dimeric forms of 80% E, in which a unit of 80% E is coupled by a linking means to another unit of 80% E from the same or different flavivirus.

WO02/072036 relates to other forms and applications of the WNV E protein, including certain specific regions such as 121-139 and 288-301. The experimental work describes cloning of the 1-406 region, which is expressed as a fusion protein, either with malB or thioredoxin as carrier.

WO04/016586 describes the E protein domain III as useful in therapy and diagnostics of WNV. In the example given on page 41, this domain comprises residues 296-415 of the WNV E protein. In another example, this domain constitutes residues 300-395.

### SUMMARY OF THE INVENTION

WNV antigen is desirably designed to allow for high yield on expression while retaining the antigenicity necessary to capture and detect antibody circulating in the blood of WNV infected patients. On this basis, the present invention provides, as antigen, an E protein fragment that consists of residues 1-428 of WNV E protein. The E protein fragment can be provided in the form of a protein also comprising a fusion component, such as an affinity tag useful in purification, or a cleavable carrier useful, for instance, to improve expression levels. Moreover, the system used to achieve expression can encode an N-terminal methionine for intracellular accumulation, and/or an N-terninal signal to permit secretion of the E protein fragment or fusion into the growth medium of the production host.

In one of its aspects, the present invention therefore provides a polypeptide consisting essentially of residues 1-428 of the WNV E protein. In embodiments, the polypeptide further comprises flanking protein residues that serve any of a variety of functions such as to facilitate expression, purification or immobilization on a solid phase.

In another aspect, the present invention provides a polynucleotide that encodes the E protein fragment or fusion protein, an expression vector in which the polynucleotide is incorporated for expression, and a host comprising the expression vector, for production of the E protein fragment or fusion protein.

In a further aspect, the invention provides a process for preparing an antigen useful to detect circulating antibody to WNV, comprising the step of culturing the host, and obtaining the WNV E protein fragment or fusion produced thereby.

In a further aspect, the invention provides an assay for detecting circulating WNV antibody, comprising the step of mixing a sample suspected of containing WNV antibody with the WNV E protein fragment or fusion, and determining the formation of complexes between said E protein and antibody, thereby to diagnose WNV infection. The assay can usefully detect antibodies of the IgG class, of the IgA class, and of the IgM class.

These and other aspects and embodiments of the invention are described in greater detail below with reference to the accompanying drawings, in which:

### BRIEF REFERENCE TO THE DRAWINGS

FIG. 1 shows an SDS-PAGE analysis of expression products from an E. coli Rosetta 2 (DE3) strain transformed with pET21b incorporating DNA encoding a polypeptide (ABCIII) that incorporates the WNV E(1-428) protein, where the lanes are: (1) un-induced culture, (2) 3 hours after induction, (3) 5 hours after induction, and (4) overnight culturing after induction; and

FIG. 2 shows a Western blot of the WNV E protein (ABCIII) produced as above and detected using antibody 6B6C-1 at 4.11 mg/ml as capture and goat anti-mouse IgG Fc gamma labeled with horseradish peroxidase, where lanes 1 & 5 are for purified recombinant protein incorporating WNV E(1-312) (35.8 kDa) which is shown for comparison. All other lanes are for protein that incorporates WNV E(1-428) for two clones 5 and 6 (lanes 2-4 and 6-8 respectively) with no induction (lanes 4 and 8), 3 hours (lanes 3 and 7), and 5 hours (lanes 2 and 6) after induction.

### DETAILED DESCRIPTION OF THE INVENTION

In the present invention, there is provided a polypeptide comprising a fragment of the WNV E protein, wherein the E protein fragment consists of residues 1-428. The E protein fragment is essentially free from other WNV E protein residues. This 1-428 fragment of the WNV E protein has the amino acid sequence of residues 2-429 of SEQ ID NO. 1. This particular E protein sequence is found within the WNV isolate designated NY99-flamingo-382-99, the complete genomic sequence for which is provided as AF196835. The protein sequence has been designated AAF20092.2. The strain is described by Lanciotti et al in Science, 1999, 286(5448):2333, incorporated herein by reference. The present invention further embraces variants of this E protein fragment sequence that incorporate for instance from 1 to 5 amino acid insertions, deletions or substitutions. In one embodiment, a substitution is a conservative amino acid substitution. In preferred embodiments, the variants are naturally occurring variants of the E protein that occur in different West Nile Virus strains that are infective for target hosts, and particularly humans, as well as birds and mammals including wildlife, pets and livestock. These variants include particularly those E protein variants that compete with the E protein fragment within SEQ ID NO. 1 for binding to an antibody that recognizes the E protein fragment within SEQ ID NO. 1. In a specific embodiment, the variants include those that occur naturally and compete with the present E protein fragment for binding to monoclonal antibody MAb 6B6C (see infra), or for binding to monoclonal antibody 5H10 which recognizes domain III of the E protein and is available from BioReliance (catalog #80-524). Naturally occurring E protein variants include those expressed by the West Nile Virus strains designated EthAn4766, 385-99, Kunjin MRM16, Golblum, TL4-4, DakAnMg, and 804994. Specific substitutions exhibited by these variant strains, and embraced by the present invention include one or more of L-310-V, V-337-I, A-369-S, V-371-I, L-375-I, E-390-D, E-428-F and H-398-I.

In addition to the E protein fragment 1-428 or a variant thereof, the present polypeptide suitably incorporates additional residues useful in the production and/or purification and/or diagnostic use of the present E protein fragment. In one embodiment, the polypeptide comprises an N-terminal amino acid or peptide extension. In a specific embodiment, the polypeptide comprises the present E protein fragment and an N-terminal methionine, to permit the E protein fragment to be produced by expression of a corresponding polynucleotide bearing an initiating methionyl codon. In the alternative, the polypeptide may be produced initially as a fusion bearing an N-terminal signal peptide to enable secretion of the E protein fragment from the production host. Numerous secretion systems and signal peptides are known for this purpose, and can be selected with regard to the particular species of host chosen for production of the protein.

The E protein fragment may alternatively comprise an N-terminal and/or C-terminal carrier peptide that is either cleaved from the E protein fragment before commercial use, or is retained in the commercial product to provide an advantage in terms of its therapeutic or diagnostic end-use. Carrier peptides that are cleaved prior to use of the E protein fragment include those that provide an advantage in terms of selection of transformants, elevation of expression levels, and those that provide an advantage in purification. To enhance expression levels, the carrier peptide may include peptides known to be expressed at very high levels when a given host/expression system is used. For instance, in E. coli, the carrier peptide may be growth hormone or a fragment thereof, β-galactosidase, mal B, thioredoxin, and the like. The cleavable carrier peptides can further include those which assist in purification of the E protein fragment, such as the well known HisTag system, and glutathione-S-transferase and the like. Such carrier peptides are fused to the E protein fragment through a peptide linker that is cleavable, using either chemical means such as metals like nickel, heat or pH adjustment or enzymatically using for instance factor Xa, clostripain, papain, tryptic enzymes and the like. Desirably, the cleavage system chosen for use yields a polypeptide product that is free from residues that are artifacts of the production process. The cleavable peptides can include the single N-terminal methionine, which is eliminated by some E. coli hosts during production thereof.

The N-terminal or C-terminal carrier peptides can further comprise peptides that are retained in the polypeptide product comprising the E protein fragment. Such carrier peptides can include those that enhance immunogenicity, for purpose of raising antibodies in a recipient host. Immunogenicity-enhancing carrier peptides include KLH and the like. Thus, the present invention further provides an immunogen comprising the present WNV E (1-428) protein fused to an immunogenicity-enhancing peptide, such as KLH. Other such carrier peptides include those useful for in vitro diagnostics. These include peptides that assist in the anchoring of the E protein fragment to a solid phase material, such as a bead, microwell or a substrate such as a sheet or strip of nitrocellulose. Particularly suitable for this purpose is a carrier peptide that is streptavidin or a biotin-binding fragment or mutant thereof.

In another specific embodiment, the polypeptide is provided in biotinylated form, for diagnostic use particularly in combination with a substrate having a streptavidin anchor immobilized on a solid support.

Thus, in embodiments of the present invention, the polypeptide comprises a WNV E protein fragment consisting of residues 2-429 of SEQ ID No. 1 and, fused to either the N-terminus or to the C-terminus thereof, a carrier peptide. The carrier peptide can be fused therewith either directly or through a cleavable peptide linker. The carrier peptide is selected to provide a processing or end-use advantage, but is also selected so as not to eliminate or substantially reduce the avidity of binding between the E protein fragment and WNV E protein antibody circulating in the blood of subjects infected with West Nile virus. Typically but not essentially, the carrier peptide will comprise from 1 to up to 200 or more amino acids, more usually from 1 to about 50, 25, 20, 15, 10 or 5 amino acids.

In a specific embodiment of the invention, the polypeptide comprises WNV E protein fragment 1-428 consisting of residues 2-429 of SEQ ID NO. 1, and optionally incorporates an N-terminal methionine to provide a protein of residues 1-429 of SEQ ID No. 1. In another specific embodiment, the polypeptide comprises WNV E protein fragment 1-428, an N-terminal methionine, and a C-terminal extension, or carrier peptide, comprising a HisTag (a hexahistidine sequence). This protein has the full amino acid sequence of SEQ ID No. 1.

The polynucleotides encoding such polypeptides constitute another aspect of the present invention. In one embodiment, the WNV E protein fragment 1-428 is encoded by a polynucleotide having the nucleic acid sequence of residues 4-1287 of SEQ ID No. 2. In another embodiment, the full polypeptide of SEQ ID No. 1, bearing the N-terminal methionine and comprising the C-terminal HisTag is encoded by the full nucleic acid sequence of SEQ ID No. 2 (residues 1-1311, the final codon representing a stop codon) Also embraced by the present invention are coding equivalents of such polynucleotide sequences, including RNA sequences and sequences that incorporate codons synonymous with those set out in SEQ ID No. 2.

To produce the present polypeptide, the polynucleotides coding therefor can be expressed from any desired cellular host having the polynucleotide incorporated expressibly therein, i.e., in which the polynucleotide is linked to, and under the expression control of, a promoter and other elements required for expression. For this purpose, the polynucleotide can be incorporated for within any of the wide variety of expression and secretion vectors that have been developed for use with a given production host. It is not essential, for diagnostic purposes, that the present polypeptides incorporate any glycosylation. Thus, for convenience, the present polypeptides can be produced in an E. coli host for which numerous expression and secretion systems are currently commercially available. These include the pET system available from Novagen, including particularly the pET21 system, and systems based for instance on such promoters as lac, trp, tac, T7, 1pp, and the like. Of course, the polypeptides can also be produced in other bacterial systems as well as eukaryotic systems including mammalian systems, based on such hosts as Saccharomyces, Pichia, Aspergillus, Trichoderma, as well as mammalian cells such as COS, CHO and the like.

The culturing of the host transformed genetically to express the present polypeptide will be done under conditions conducive to the viability and expansion of the host as well as to the activation of the promoter controlling expression of the incorporated polynucleotide. Typically, supplements required to activate or de-repress the promoter will be added when the culture reaches early to mid-log phase, and the polypeptide produced upon culturing will be recovered either directly from the spent culturing medium, in the case where a secretion system is used or, in the case where intracellular production is utilized, from the total protein released following cell lysis. In those cases where inclusion bodies are formed intracelluarly, the protein will be solubilized before polypeptide recovery, all in accordance with procedures well established for these purposes.

The recovered polypeptide can be purified also using standard practise in the art, including enrichment based on size and charge, as well as enrichment based on binding affinity. Such purification can occur either before or after any cleavable carrier peptide is removed. For those polypeptides that incorporate a carrier peptide included to assist in purification, purification can occur on an affinity column packed with a solid phase that incorporates a ligand for the carrier peptide, or for the E protein fragment itself. Such affinity ligands can include, for instance, immobilized antibody to the E protein, such as the 6B6C antibody described infra. Polypeptides that incorporate the HisTag can be purified on a column of immobilized nickel using commercially available reagents and protocols well established for this purpose.

In one embodiment, the polypeptide is provided in lyophilized form, together with standard bulking agents and/or excipients, for longer-term storage.

Once obtained, the present polypeptides are useful particularly in the performance of in vitro diagnostic assays for the detection of WNV E protein antibody that circulates in the blood of patients afflicted with West Nile disease. Antibodies that are of the IgM class and that bind to the E protein appear in the circulation within several days following the first symptoms of infection. In one embodiment of the invention, there is provided a method for detecting antibody to the West Nile virus, comprising the step of combining a polypeptide of the present invention with a sample suspected of comprising WNV antibody, and determining whether any complex has formed, the formation being indicative of the presence of the antibody.

In particular embodiments of the invention, the present polypeptides are used particularly to detect the presence of WNV antibody in a sample extracted from a patient suspected of WNV infection. In embodiments, the sample is whole blood or a whole blood fraction, such as plasma or serum. In the alternative, the sample is urine, cerebrospinal fluid, or any other body fluid in which antibodies reside. In general, the present polypeptide is mixed with the sample, and formation of a complex between the polypeptide and any antibody present in the sample is then detected, the formation of complex being indicative of the presence of WNV antibody in the sample.

In other particular embodiments, the present polypeptide is immobilized on a solid phase, either directly or through a linkage such as a biotin/streptavidin coupling, or by affinity to an immobilized ligand such as an antibody to the E protein or an antibody or other ligand to any carrier protein present on the polypeptide. The immobilized polypeptide then serves as a ligand to capture and immobilize any WNV E protein antibody present in a sample incubated therewith. Following such incubation, and washing to remove excess or unbound protein, the captured WNV antibody is detected by incubation with an appropriately labeled ligand therefor. The labeling can be achieved using a binding or labeling system, in which for instance, antibody to human IgM (or IgM antibody to a different species in the case where infection in non-humans is under investigation) is provided in a form labeled with a detectable moiety, such as colloidal gold, a fluorophor or lumiphor, or an enzyme for a colorimetric substrate is available). In a specific embodiment, this “detector” reagent is provided as colloidal gold-labeled antibody to human IgM, or to human IgG.

In one embodiment, such a diagnostic assay is performed on the surface of a prepared substrate such as nitrocellulose. In particular, and according to one embodiment of the present invention, there is provided a substrate useful in performing a lateral flow immunoassay for detection of WNV IgM antibody in a sample. The substrate comprises a reagent immobilized at a downstream test site for capturing a labeled reagent complex formed upstream thereof. The immobilized capture reagent can, for instance, be an immobilized antibody to human IgM, such as goat anti-human IgM antibody. Alternatively, the immobilized capture reagent can be an equivalent biotinylated antibody, bound to a streptavidin anchor immobilized on the substrate. To label any WNV IgM present in the sample, the substrate further comprises, at an upstream site, deposited mobile reagents that can include the E protein fragment of the present invention, and a labeled antibody thereto, such as gold labeled 6B6C MAb. The mobile reagents are positioned to be entrained within the sample as it migrates along the strip toward the test line, so that a labeled complex first forms between any WNV IgM in the sample and the E protein of the present invention, which in turn is bound to and labeled with the anti-E protein antibody such as the gold labeled 6B6C antibody. The anti-IgM antibody immobilized at the test line then captures the migrating complex. Accumulation of the labeled complex at the immobilized antibody site is indicative of the presence of WNV IgM antibody in the sample, and diagnostic for WNV infection. In the absence of WNV IgM, the above labeled complex does not form, the label does not accumulate at the test line, and the result is negative for WNV IgM.

In specific embodiments, the substrate is nitrocellulose. In still other embodiments, the substrate is contained within a housing that provides an inlet for introducing sample, and a downstream window permitting the user to view any detector reagent captured by the immobilized polypeptide.

In other specific embodiments, the polypeptide is the E protein fragment of SEQ ID No. 1, comprising an N-terminal methionine optionally with a C-terminal HisTag or other carrier peptide, the detector reagent is colloidal gold-labeled antibody to WNV E protein, and the sample is whole blood taken from a human or other species such as bird, horse, etc., suspected of being infected with WNV.

To provide a control for the assay, the substrate may further comprise, as a control, a reagent that is immobilized downstream from the capture reagent and that binds to the labeled detector reagent, whereby the presence of label at the immobilized control indicates that sample and mobile reagent have migrated successfully beyond the immobilized capture reagent.

It will be appreciated that the present polypeptides are useful in numerous other assay formats. The polypeptides themselves can serve as the detector reagent, and for this and other purposes can be provided in labeled form, i.e., bearing a label that is or can be detected, e.g., visually or instrumentally. Such labels including colloidal metals including colloidal gold, enzymes including horseradish peroxidase and alkaline phosphatase, and the like. For example, one suitable assay format entails the immobilization antibody to Fc epsilon (the constant domain of IgM), which captures IgM present in the sample, and labeled WNV E protein to identify captured IgM that is specific to WNV.

It is to be appreciated that the polypeptides are useful also in assays designed to detect IgG to WNV. This is achieved using anti-human IgG antibody as the capture reagent instead of anti-human IgM antibody.

It is also to be appreciated that assays utilized the present polypeptides can be performed using for instance the MAC-ELISA format, which utilizes trays of microwells as the solid phase to which anti-IgM is immobilized, that are incubated with sample and then washed. Bound human IgM that is specific to WNV is then detected by adding a complex comprising WNV antigen complexed with anti-antigen antibody that is labeled with horse radish peroxidase and revealed by color change after substrate introduction.

From the results that follow, it will be appreciated that the E protein fragment 1-428 of the present invention is particularly well adapted to be produced at usefully high production levels and to yield assay results that are clinically superior.

### EXAMPLE 1

### Cloning and Expression of WNV E (1-428)

Polynucleotides encoding the West Nile Virus E protein are available from a variety of different sources, and can be amplified therefrom using conventional techniques. In the following example, the WNV E protein is cloned from an available source. This is the COS-1 cell line (WNC2 clonal—selected cell line) that is stably transformed with WNV PrM, M & E proteins genes. This cell line is available from the Centres for Disease Control and Prevention of the U.S. Public Health Services.

Using the Puregene Kit, genomic DNA was purified from these above COS-1 cells harvested from cell culture plates. To amplify the WNV-E cDNA with PCR from genomic DNA correctly and to sequence it easily, the genomic DNA was amplified in 4 pieces. Since the restrict endonucleases NcoI, BsgI, KasI, PshAI & Xhol can cut the full length E cDNA into 4 fragments, 4 pairs of primers were designed with the appropriate enzyme site in each. Using the genomic DNA as template, the above primer pairs and the pfu DNA polymerase with PCR, there were amplified the following cDNA fragments: fragment A: NcoI-BsgI, fragment B: BshI-KasI, fragment C: KasI-PshI, and fragment D: PshI-XhoI. After subcloning them into pre-digested pCR-Script Amp Sk(+) vector(ps) and transforming into E. coli, then amplifying & purifying the plasmid DNA, sequencing confirmed the correct sequence and orientation.

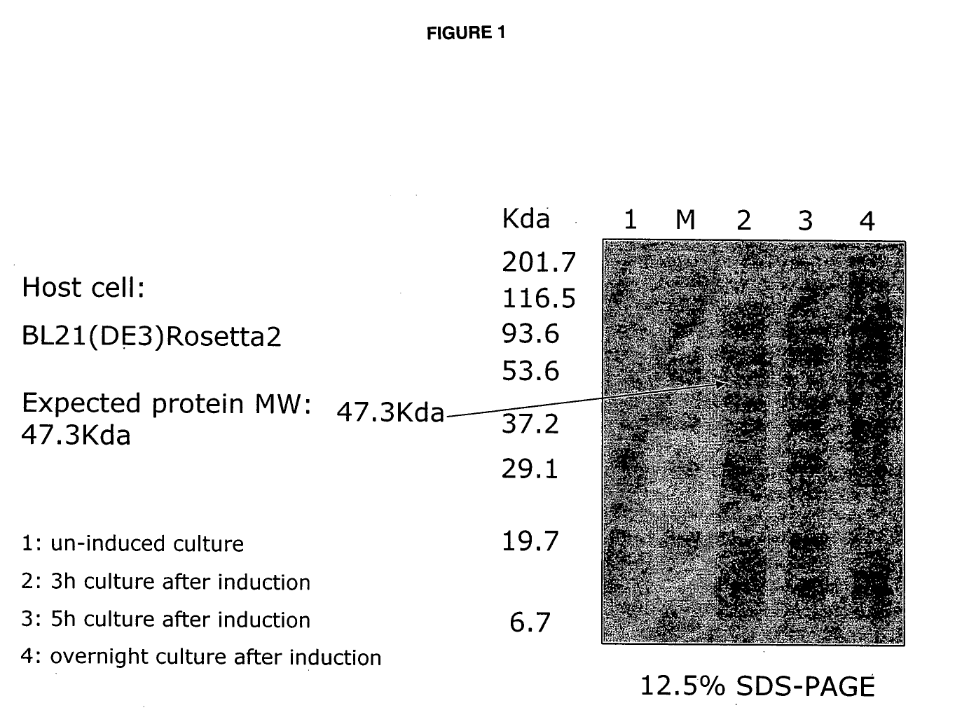

To allow for subsequent comparison of expression levels, the resulting full length clone of WNV E protein was then constructed from the polynucleotide fragments that encode the four separate domains of the E protein, designated A, B, C and D respectively, and reviewed for instance by Mandl et al, J. Virol., 1989, 564-571. The resulting clones were then integrated for expression into the T7 promoter-driven pET21b expression system available from Novagen, and transformed for production into a Rosetta 2 (DE3) strain of E. coli, also available from Novagen (catalog number 71397-4), which was then selected, cultured and induced according to the supplier's instructions. Briefly, cells were grown overnight at 37 C on LB plates containing ampicillin and chloramphenicol for selection, and surviving cells were collected by scraping into 3 ml of LB medium and culturing for a further one hour at 37 C. Cultures were then induced using 3 ul of 1M IPTG, and 200 ul samples were extracted at various time points (see FIG. 1).

To generate samples for analysis, solubilization of the E protein from inclusion bodies was achieved in the usual way. Briefly, samples were centrifuged, cell pellets were resuspended in 1× sample buffer containing B-mercaptoethanol, SDS and bromophenol. After boiling for two minutes, sonicating and centrifuging the treated sample, the supernatants were loaded as 30 ul samples onto the SDS-PAGE, and run.

The expression results were as follows, where “+” indicates expression, and “−” indicates non-detectable expression:

| Vector | Sequence | Expression |
|---|---|---|
| WNE-A/pET21 | 1–101 | ++ |
| WNE-B/pET21 | 92–182 | −−− |
| WNE-C/pET21 | 174–323 | ++ |
| WNE-D/pET21 | 318–502 | −−− |

Using the same restriction endonucleases approach, various fragment combinations were excised and combined, and expressed using the same system noted above, with the following results:

| Vector | Sequence | Expression |
|---|---|---|
| WNE(A + B)/pET21 | 1–182 | −−− |
| WNE(A + B + C)/pET21 | 1–319 | ++ |
| WNE(A + B + C + D)/pET21 | 1–502 | + (by Western Blot only) |

Since WNE(A+B+C) expressed very well, whereas WNE-D and WNE(A+B+C+D) could not be expressed, it was clear that the D domain or a portion thereof may be responsible for the yield depression. Yet, this domain incorporates many features believed to be important to the function and immune recognition of the protein, including several neutralizing epitopes, WNV subtype-specific epitopes and secondary structure believed to be important as T cell antigenic determinants. It was accordingly necessary to incorporate certain domain D features without significant loss of expression.

This was accomplished by eliminating codons 429-502 of the E protein, to produce a plasmid designated WNE(A+B+C(III)). The coding region of this plasmid has the polynucleotide sequence of SEQ ID No. 2, which encodes an N-terminal methionine residue, residues 1-428 of the WNV E protein, a linking dipeptide sequence LE and the hexahistidine sequence constituting the HisTag and a stop codon. Expression levels were at least equivalent to those levels seen with the ABC construct, and far exceeded those seen with the ABCD construct.

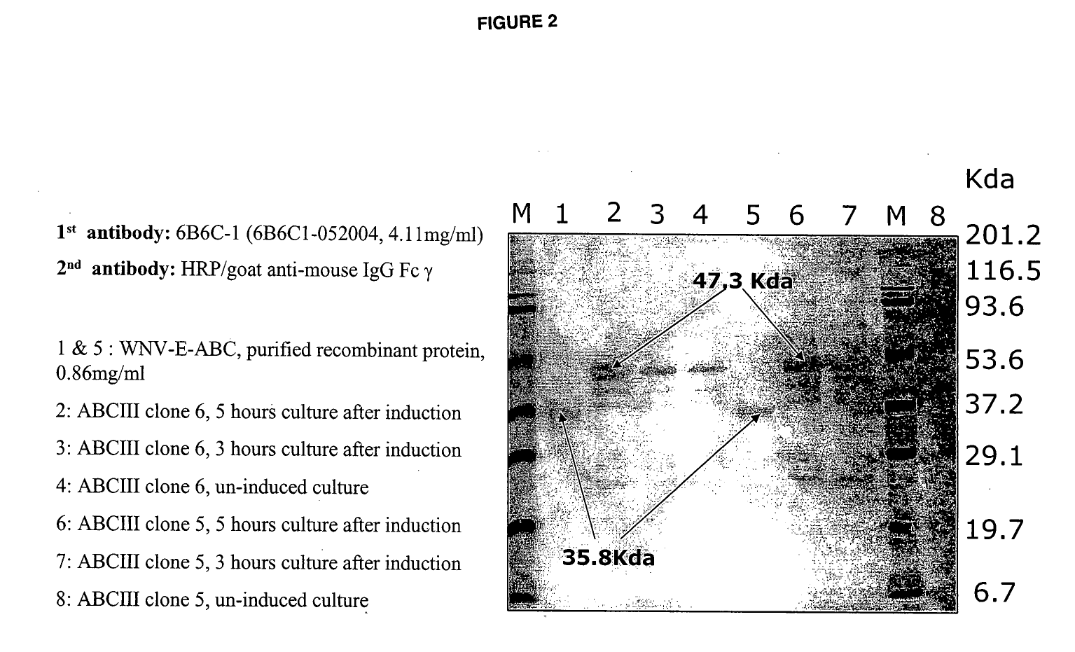

Western blot analysis of the polypeptide of SEQ ID No. 1 incorporating the E(1-428) fragment, is shown in FIG. 2. The first antibody (4.11 mg/ml) was the 6B6C-1 mouse antibody to the E protein available from the CDC. The second, detector antibody was goat anti-mouse IgG Fcγ labeled with horseradish peroxidase. Analysis confirms the presence of the expected 47.3 kDa protein, which is also confirmed by SDS-PAGE analysis of culture at different culturing time points with and without induction (FIG. 1).

### EXAMPLE 2

### Use of the E Protein for in Vitro Diagnostics

A lateral flow immunoassay format was adopted using the device similar to that commercialized by Spectral Diagnostics Inc. under the trade name Cardiac STATus. This device comprises a nitrocellulose substrate having an upstream site on which reagent pads are mounted, to receive sample. For purposes of detecting IgM to WNV, one sample pad is impregnated (deposited and lyophilized) with gold-labeled 6B6C-1 antibody, as detector antibody (available from the Centre for Disease Control, and first described by Roehrig et al in Virology, 1983, (128):118-126) and mounted above a second reagent pad impregnated with the WNV E protein fragment of SEQ ID No. 1. The pads are stacked above and in flow communication with the nitrocellulose substrate, which bears a test line defined by an immobilized goat anti-human antibody (Jackson Laboratories) reactive with any human IgM in the sample. Downstream of the test line, the nitrocellulose substrate also provides a control line constituted by immobilized antibody that captures any unreacted gold-labeled detector antibody. Patient serum/plasma is added to the top of the stacked reagent pad, and migrates toward and across the control line for accumulation at a distal, absorbent pad, bringing reagents and reaction complexes into contact with the test line, where accumulation of a visible pink colour indicates the sample contained WNV IgM.

Results indicate there is no cross-reactivity with any of the tested other virus, including HSV1, HSV2, CMV, syphilis, EBV IgM, EBV IgG, HIV, JEV, SLEV, dengue, HBsAg, HCAb or California encephalitis. Sensitivity was 95% in agreement with FOCUS Elisa for positives, 99% for negatives, and 97% overall.

The present E protein thus represents a WNV antigen that can be expressed at relatively high levels, while retaining utility as a reagent in the sensitive, specific and rapid detection of circulating WNV IgM.

What is claimed is:

1. A polypeptide comprising a fragment of the E protein of WNV, said WNV E protein fragment consisting of residues 2-429 of SEQ ID No. 1.

2. The polypeptide according to claim 1, further comprising a carrier protein.

3. The polypeptide according to claim 2, wherein the carrier protein is a HisTag.

4. A polypeptide according to claim 3, having SEQ ID No. 1.

5. A polynucleotide encoding the polypeptide according to claim 1.

6. A polynucleotide encoding the polypeptide according to claim 4, the polynucleotide comprising the nucleic acid sequence of SEQ ID No. 2.

7. An expression vector comprising said polynucleotide encoding the polypeptide according to claim 1, linked operably with DNA enabling expression thereof.

8. A host cell having the polynucleotide according to claim 5 incorporated expressibly therein.

9. A process for producing a West Nile virus antigen useful to detect antibody to WNV in a patient sample, comprising the step of culturing the host cell according to claim 8.

10. An in vitro diagnostic assay useful to detect WNV antibody present in a patient sample, the assay comprising the step of combining the patient sample with the polypeptide according to claim 1, and detecting the formation of complex thereby to detect the presence of WNV antibody in the sample.

11. An assay according to claim 10, wherein said complex is detected using labeled antibody to said WNV antibody

12. An assay according to claim 10, wherein said polypeptide is immobilized either directly on a substrate or indirectly through a ligand immobilized on a substrate.

13. A substrate useful in a lateral flow immunoassay for the detection of WNV antibody present in a patient sample, the substrate comprising a sample addition site and a downstream test site, wherein the sample addition site comprises, as mobile reagents, a polypeptide according to claim 1 and a non-human antibody that binds WNV E protein and comprises a detectable label, and a test site comprising an immobilized reagent that binds human IgM antibody.

14. A method for performing a lateral flow immunoassay for the detection of WNV antibody in a patient sample, comprising the steps of (1) obtaining a substrate as defined in claim 13, (2) adding sample to the sample test site and, after a period sufficient for reagents to migrate with the sample to a position downstream of the test site, (3) determining the presence of label at the test site.

15. The method according to claim 14, wherein the patient sample is selected from whole blood, serum and plasma.


SEQUENCE LISTING (2 sequences)

SEQ ID NO: 1 - protein, 437 aa, Artificial Sequence
  synthetic
MFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFLEHHHHHH

SEQ ID NO: 2 - DNA, 1314 bp, Artificial Sequence
  synthetic
atgtttaattgtttaggaatgagcaacagagacttcttggaaggagtgtctggagcaacatgggtggatttggttctcgaaggcgacagctgcgtgactatcatgtctaaggacaagcctaccatcgatgtgaagatgatgaatatggaggcggccaacctggcagaggtccgcagttattgctatttggctaccgtcagcgatctctccaccaaagctgcgtgcccgaccatgggagaagctcacaatgacaaacgtgctgacccagcttttgtgtgcagacaaggagtggtggacaggggctggggcaacggctgcggactatttggcaaaggaagcattgacacatgcgccaaatttgcctgctctaccaaggcaataggaagaaccatcttgaaagagaatatcaagtacgaagtggccatttttgtccatggaccaactactgtggagtcgcacggaaactactccacacaggttggagccactcaggcagggagattcagcatcactcctgcggcgccttcatacacactaaagcttggagaatatggagaggtgacagtggactgtgaaccacggtcagggattgacaccaatgcatactacgtgatgactgttggaacaaagacgttcttggtccatcgtgagtggttcatggacctcaacctcccttggagcagtgctggaagtactgtgtggaggaacagagagacgttaatggagtttgaggaaccacacgccacgaagcagtctgtgatagcattgggctcacaagagggagctctgcatcaagctttggctggagccattcctgtggaattttcaagcaacactgtcaagttgacgtcgggtcatttgaagtgtagagtgaagatggaaaaattgcagttgaagggaacaacctatggcgtctgttcaaaggctttcaagtttcttgggactcccgcagacactggtcacggcactgtggtgttggaattgcagtacactggcacggatggaccttgcaaagttcctatctcgtcagtggcttcattgaacgacctaacgccagtgggcagattggtcactgtcaacccttttgtttcagtggccacggccaacgctaaggtcctgattgaattggaaccaccctttggagactcatacatagtggtgggcagaggagaacaacagatcaatcaccattggcacaagtctggaagcagcattggcaaagcctttacaaccaccctcaaaggagcgcagagactagccgctctaggagacacagcttgggactttctcgagcaccaccaccaccaccactga